(19) United States
(12) Patent Application Publication
(10) Pub. No.: US 2026/0137520 A1
(43) Pub. Date: May 21, 2026

(54) HYDRAULIC DEPLOYMENT OF ENDOBRONCHIAL CHECK VALVE

(71) Applicant: GYRUS ACMI, INC. D/B/A OLYMPUS SURGICAL TECHNOLOGIES AMERICA, Westborough, MA (US)

(72) Inventor: Joey Magno, Dudley, MA (US)

(21) Appl. No.: 18/954,060

(22) Filed: Nov. 20, 2024

Related U.S. Application Data
(60) Provisional application No. 63/601,501, filed on Nov. 21, 2023.

(51) Int. Cl. A61F 2/24; A61B 1/00; A61B 1/267; A61F 2/48; A61M 25/00

(52) U.S. Cl. CPC A61F 2/2476; A61B 1/00087; A61B 1/2676; A61F 2/484; A61M 25/0082

(57) ABSTRACT

A device can include a fitting including a first aperture, a second aperture, and a valve engageable to restrict fluid travel between the first and second apertures. The device can also include an elongate flexible shaft for coupling with the fitting. A piston can be disposed within the passage and configured to travel toward the distal end of the shaft while maintaining a fluid seal between first and second sides of the piston. Upon the valve being engaged, fluid pressure received at the first aperture can be translated to drive the piston toward the distal end of the shaft.

### CLAIM OF PRIORITY

This application claims priority to US Provisional Application Ser. No. 63/601,501, filed on Nov. 21, 2023, which is incorporated by reference herein in its entirety, and the benefit of priority of which is claimed herein.

### BACKGROUND

Bronchoscopy is a procedure that allows doctors to examine the breathing passages and deliver treatments to the lungs. During bronchoscopy, an instrument called a bronchoscope is inserted through the nose or mouth and guided down the throat into the lungs. This allows the doctor to view the airways, e.g., via a video monitor or other imager, and perform procedures. One condition that can be treated with bronchoscopy is severe emphysema, which involves damage to the air sacs in the lungs.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depicts an example of an endobronchial check valve for placement within a lung of a patient.

FIG. 1B depicts an example of an endobronchial check valve arranged within an endobronchial implant placement device, the endobronchial check valve in an undeployed position.

FIG. 1C depicts an example of an endobronchial check valve deploying from an endobronchial implant placement device, the endobronchial check valve in an intermediate position.

FIG. 1D depicts an example of an endobronchial check valve as deployed from and placed by the endobronchial implant placement device.

FIG. 1E depicts an example of an endobronchial check valve as placed within a lung of a patient.

FIG. 1F depicts an example of an endobronchial check valve as placed within a lung of a patient.

FIG. 2A depicts an example of an endobronchial check valve placement device.

FIG. 2B depicts an example of an endobronchial check valve placement device paired with a source of external pressure.

FIG. 3A depicts an example of the distal portion of the elongate flexible shaft of an example of an endobronchial implant placement device during motion of the piston within the passage of the shaft.

FIG. 3B depicts an example of the distal portion of the elongate flexible shaft during motion of the piston within the passage of the shaft.

FIG. 3C depicts an example of the distal portion of the elongate flexible shaft during motion of the piston within the passage of the shaft.

FIG. 4 is a flowchart describing a method of placing an endobronchial check valve via a deployment device.

### DETAILED DESCRIPTION

Chronic obstructive pulmonary disease (COPD) and emphysema are diseases that cause reduced lung function and difficulty breathing due to damage to lung tissue. Patients with severe cases may have hyperinflated lung lobes that compress adjacent healthier lung tissue, preventing full expansion and reducing the ability to exchange gases. One technique to treat advanced emphysema is to place an airway bypass implant in the airway leading to the hyperinflated lung lobe, isolating the damaged region. This allows the healthier lung tissue to fully expand and function properly. One such airway bypass implant is an implantable endobronchial check valve, also called a “Spiration Valve System (SVS)” valve which acts as a one-way valve to allow air and fluids to exit the isolated diseased portion of the lung during exhalation, while preventing air from entering during inhalation. This enables the hyperinflated lung region to deflate over time.

The implantable endobronchial check valve can be deployed into the target airway using a catheter loaded with the valve. The catheter has a handle assembly on the proximal end and a working channel on the distal end that interfaces with the working channel of a bronchoscope. This allows navigation of the catheter and valve to the target airway location under bronchoscopic visualization. Certain endobronchial implant delivery catheters can involve a mechanical actuation system to deploy the endobronchial check valve. For example, this can involve several injection molded plastic components in the handle assembly that interface with a stabilization wire running through the catheter. Here, the wire can be connected to a piston at the distal tip of the catheter and can be used to control delivery of the endobronchial check valve out of the distal end of the catheter. Such a handle assembly and mechanical actuation system can involve manufacturing various specialized plastic and metal components, and the relative complexity of such a mechanism can present challenges in reliability, longevity, and sanitation for reuse. Therefore, there is a need for an improved endobronchial implant delivery device that assures dependable positioning of an endobronchial check valve an airway and, e.g., including fewer parts and a more direct translation of actuator force to an undeployed endobronchial check valve.

The present disclosure provides devices, systems, and methods for deploying endobronchial valves and other devices during bronchoscopy. A bronchoscopy system can include or use an actuator such as a pump. The system can also include a check valve placement device, including a fitting and an elongate flexible shaft. The fitting can include a first fitting aperture configured for fluid connection to the actuator, a second fitting aperture for fluid connection to the elongate flexible shaft, and a fitting valve selectively engageable to restrict fluid travel between the first and second fitting apertures. The elongate flexible shaft can define a passage between the second fitting aperture and a distal end of the shaft, and a piston can be disposed within the passage and configured to travel toward the distal end of the shaft. Engaging of the valve and actuation of the actuator can permit fluid travel between the first and second fitting apertures, and force from the actuator can be translated to the piston to eject an endobronchial device (e.g., an endobronchial check valve or a “Spiration Valve System (SVS)” valve) from the distal end of the elongate flexible shaft. The system facilitates safe, accurate placement of an endobronchial implant, and the system can be reusable for reloading and deployment of multiple different endobronchial implants, e.g., by resetting the piston position following each deployment.

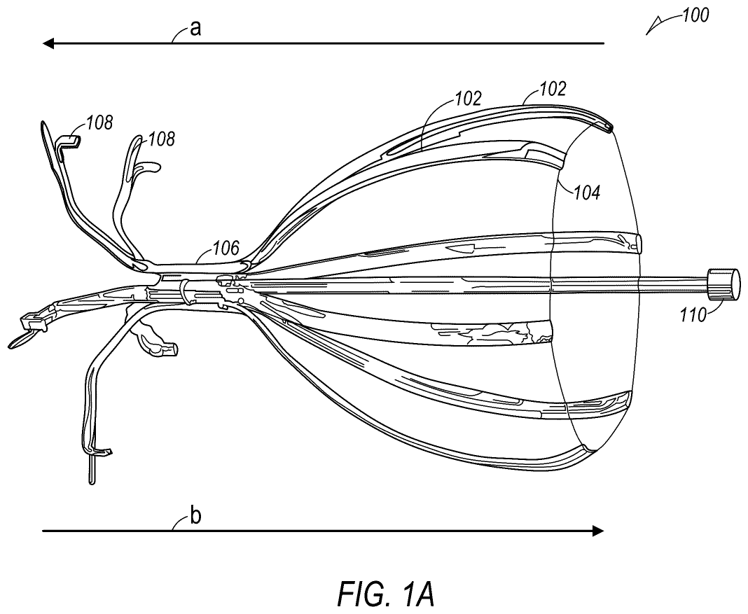

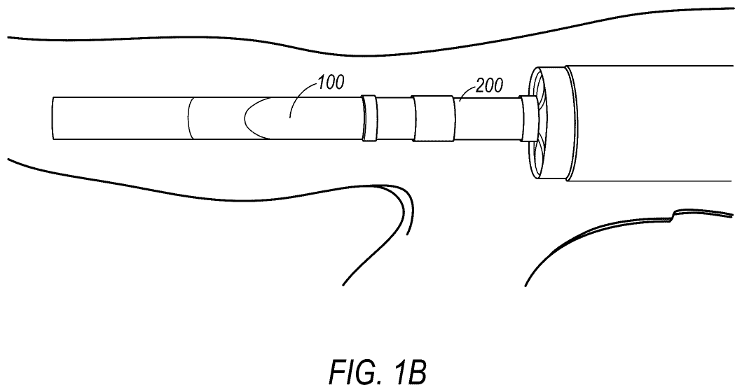

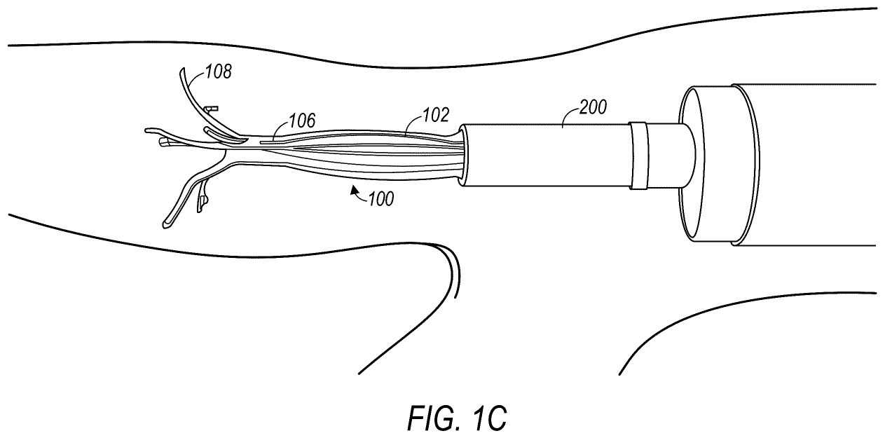

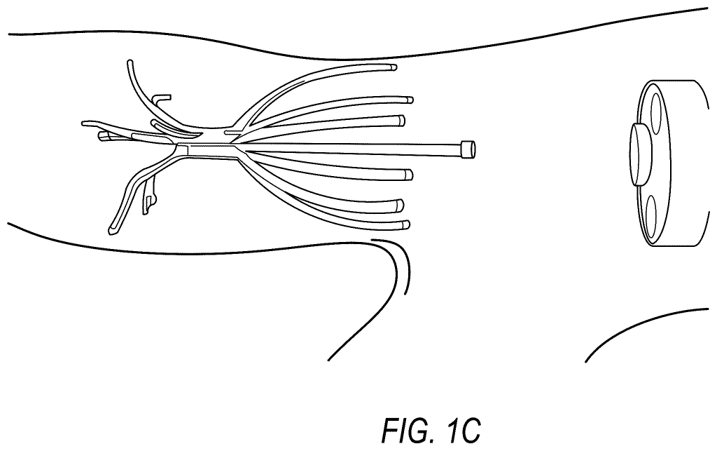

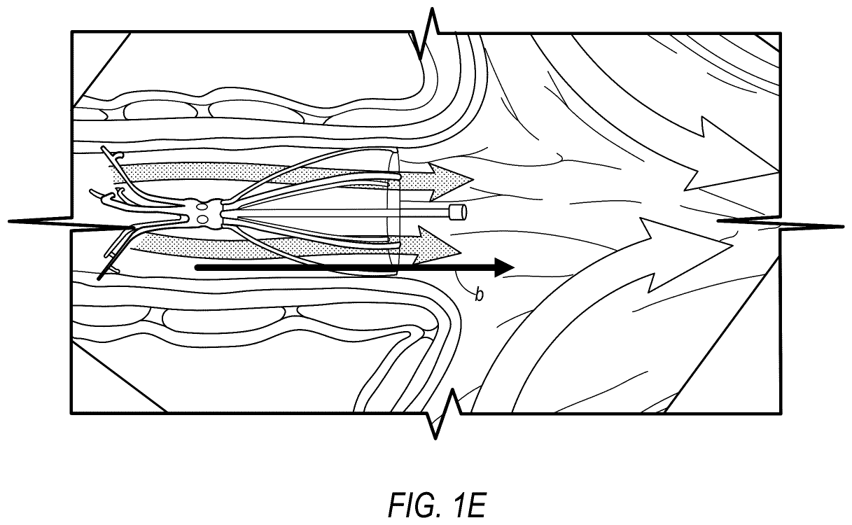

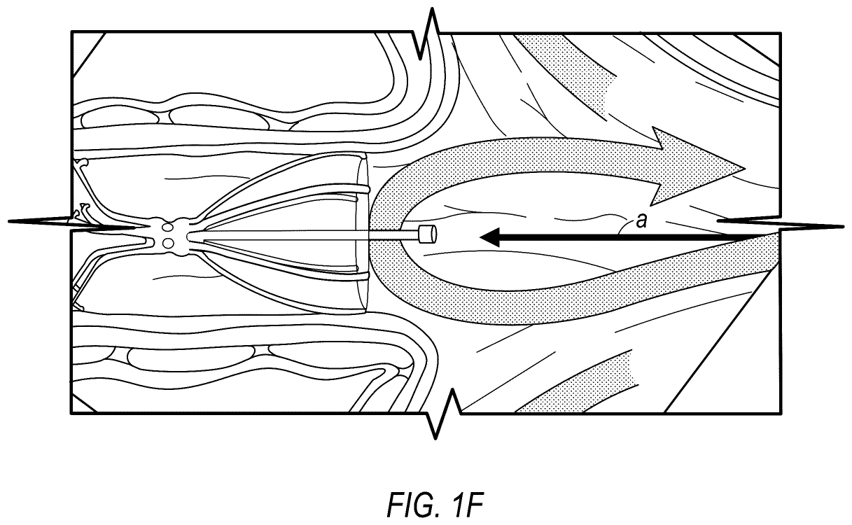

FIG. 1A, FIG. 1B, FIG. 1C, and FIG. 1D each depict an example of an implantable endobronchial check valve for placement within a lung of a patient. Herein, an implantable endobronchial check valve can also be referred to as a Spiration Valve System (SVS) valve or generally as an example of an endobronchial implant. An endobronchial check valve 100 can include a frame including plurality of ribs 102 and a covering 104 arranged over the ribs 102. The ribs 102 can extend from a valve stem 106 at a taper, such that when placed in a patient lung, the ribs 102 form an umbrella shaped taper extending outward from the stem 106 in a proximal direction b. Herein, “distal” refers to the location in which the valve or other medical device will be deployed while proximal means toward the user of a valve-placement device (e.g., a physician). Also herein, a distal direction a can refer generally to a direction of air travel into a bronchus (e.g., toward a trachea) of the patient and the proximal direction b can refer generally to a direction of air travel out of the bronchus (e.g., away from the trachea) of the patient. The ribs 102 can be sized and shaped such that upon placement of the endobronchial check valve 100 within a bronchus or lobe of a patient lung, the taper of the ribs 102 maintain an opening through which air can travel in the proximal direction a. The ribs 102 can also stabilize the endobronchial check valve 100 within the bronchus or lobe. For example, the endobronchial check valve can have, in a deployed arrangement, a greatest cross-sectional diameter within a range of about 4 millimeters (mm) and about 15 mm, or within a range of about 5 mm and about 9 mm. In an example, the greatest cross-sectional diameter can refer to a widest diameter of the umbrella-shaped taper of the ribs 102. The endobronchial check valve 100 can also include one or more anchor features or struts 108 sized and shaped to prevent migration or expectoration of the endobronchial check valve 100 after placement in the bronchus or lobe. For example, anchor features 108 can include projections to engage mucosa or other tissue along the interior of the bronchus or lobe. In an example, the endobronchial check valve 100 can also include a retrieval feature 110, such as a tether, for use in removal of the valve. An example of an endobronchial check valve is described in the Patent Cooperation Treaty (PCT) Publication Serial No. WO 2015/153507, which is incorporated by reference herein in its entirety for its teaching of endoluminal devices such as an endobronchial check valve. While deployment and placement devices are generally described herein with respect to the endobronchial check valve 100, the endobronchial implant can include stents, valves, lung reduction valves, coils, filters, embolic protection devices, balloons, augmentation devices, probes, anchors, sponges, or any other medical device, deployable or otherwise, that is configured to be loaded or introduced into a catheter or other deployment apparatus for bronchoscopy.

FIG. 1B depicts an example of an endobronchial check valve 100 arranged within an endobronchial implant placement device 200, the endobronchial check valve 100 in an undeployed position. Herein, the implant placement device 200 can also be referred to as a deployment device or deployment catheter. The endobronchial check valve 100 can be formed of a flexible material, a shape memory alloy such as nitinol, or a polymer including polytetrafluoroethylene (PFTE), polylactide (PLA), or ethylene-vinyl acetate (EVA). In the undeployed position, the endobronchial check valve 100 can be compressed or compacted such that the greatest cross-sectional diameter is less than the same in the deployed arrangement. For example, the endobronchial check valve 100 can have, in an undeployed arrangement, a greatest cross-sectional diameter within a range of about 1.5 mm and about 3 mm, or within a range of about 2 mm and about 2.7 mm. In an example, the endobronchial check valve 100 can be compressible such that the plurality of ribs 102 and one or more anchor features 108 can be compacted to a diameter less than or equal to the diameter of the valve stem 106 while still returning to their uncompressed or deployed shape and diameter when uncompacted.

FIG. 1C depicts an example of an endobronchial check valve 100 deploying from an endobronchial implant placement device 200, the endobronchial check valve 100 in an intermediate position. In an example, the endobronchial check valve 100 can be arranged within the endobronchial implant placement device such that the plurality of ribs 102 are located proximal to the valve stem 106 or the one or more anchor features 108. In an example, at the intermediate position and during deployment, the one or more anchor features 108 can be ejected from the endobronchial implant placement device 200 before the ribs 102.

FIG. 1D depicts an example of an endobronchial check valve 100 as deployed from and placed by the endobronchial implant placement device 200. Here, the deployed and placed endobronchial check valve 100 can include the plurality of ribs 102 arranged toward the proximal direction b relative to the valve stem 106 and the one or more anchor features 108 arranged toward the distal direction a. As shown in FIG. 1E and FIG. 1F, the endobronchial check valve 100 can facilitate a one-way passage of air in the proximal direction b while restricting passage of air or other fluid in the distal direction a. In doing so, the endobronchial check valve 100 can relieve over-inflation by allowing air to escape a patient lung in the proximal direction b and also prevent air from entering the lung in the distal direction a. That is, the deployed endobronchial check valve 100 can allow air to equalize between a bronchus (or lobe) of the lung having high air pressure due to disease states like emphysema or damaged airways caused by chronic injury such as fibrosis.

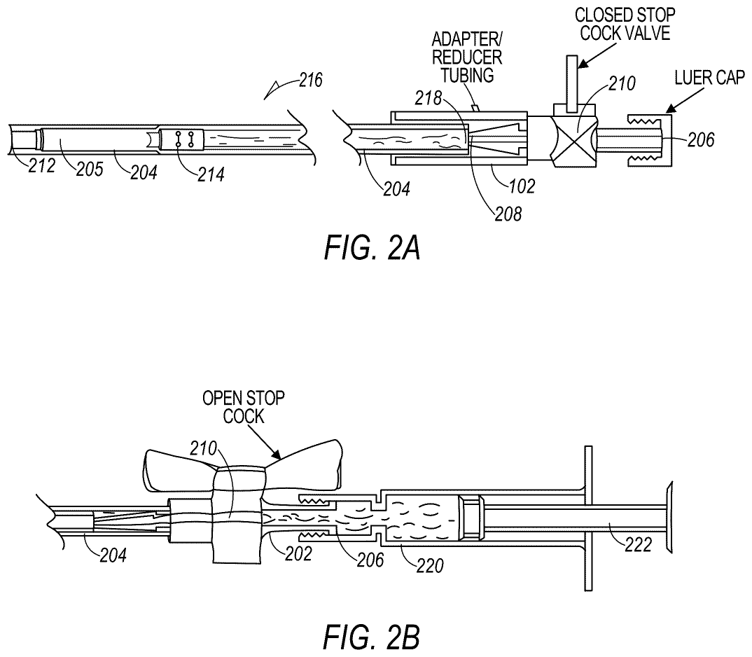

FIG. 2A and FIG. 2B depict an example of an endobronchial check valve placement device. The endobronchial check valve placement device 200 can facilitate deployment of an endobronchial implant (e.g., endobronchial check valve 100 of FIG. 1A-FIG. 1F). The endobronchial check valve placement device 200 can also control placement of an endobronchial check valve within a bronchus or lobe of a patient lung. Precision during placement of an endobronchial check valve can be highly important and can require reliable “action” of an instrument, such as including minimal play in actuation and depositing the endobronchial check valve without excess propulsion. The predominately hydraulic mechanism of the endobronchial check valve placement device 200 can provide a physician using the device with requisite control and finesse in positioning an individual endobronchial check valve at a target location, which can be challenging to accomplish with certain other mechanically actuated instruments.

The endobronchial check valve placement device can include a fitting 202 and an elongate flexible shaft 204. The fitting 202 can include a first aperture 206, a second aperture 208, and a valve 210. The fitting 202 can be formed from a relatively hard thermoplastic material, a relatively hard thermoset material, or from other materials that provide rigidity and strength to the fitting. For example, the fitting 202 can be formed from polyethylene, polyamide, nylon, polycarbonate, polypropylene, polyethylene terephthalate, acrylonitrile butadiene styrene, various metals, or hybrid materials. The fitting 202 can be made of a material that is transparent, opaque, or semi-transparent. In an example, the valve 210 can be selectively engageable or operable to restrict fluid travel between the first and second apertures 206 and 208. The valve 210 can include a stopcock, a stop valve, a toggle valve, a gate valve, a ball valve, a spool-valve, a butterfly valve, or other selectively manipulable flow control element. In an example, the valve can be operable between an open position and a closed position. Also, the valve 210 can be operable between a completely open position, a partially open position, or a completely closed position. At or near the first aperture 206, the fitting 202 can include a connection for an external source of fluid pressure, such as a syringe, a pressure pump, a liquid line, an air supply, a gas supply, a vacuum source, or other selectively controllable source of fluid pressure. For example, the first aperture 206 can be formed or disposed within a Luer tapered fitting, a single-or multi-prong connector, an O-ring-centric fluid seal, a threaded cap, or other selectively detachable attachment accessory. In an example, the first aperture 206 can be fluidly sealed by a Luer cap when not connected to the external source of fluid pressure. Also, at or near the second aperture 208, the fitting 202 can include a connection sized and shaped to couple with the elongate flexible shaft 204. For example, the second aperture 208 can be formed or disposed within a barbed connection, a reducer connection, a stepped reducer connection, a flexible tubing coupling, a threaded fitting, or other selectively attachable anchoring apparatus. The elongate flexible shaft 204 can be coupled with the aperture defined by the fitting 202 at the second aperture 208 such that vacuum or fluid pressures controlled at the first aperture 206 are conducted by the elongate flexible shaft 204 toward the distal end 212. In an example, the elongate flexible shaft 204 can comprise or include flexible tubing, rigid tubing, corrugated tubing, thermoformed tubing, spiral tubing, metal braided tubing, flexible polymer film strips, mesh grating, elastic sheeting, or other selectively pliable and shapeable forms.

In an example, the elongate flexible shaft 204 can be sized and shaped to fit within a working channel of a parent scope or a bronchoscope. For example, the elongate flexible shaft can have a relatively uniform outer diameter, along its length, within a range of about 1.5 millimeters (mm) to about 10 mm, or between about 2 mm and about 4 mm. Herein, relatively uniform outer diameter means less than +/− about 5% variation in outer diameter over the length of the elongate flexible shaft 204. The elongate flexible shaft 204 can define a passage 205 between the second aperture 208 of the fitting 202 and a distal end 212 of the shaft 204. For example, the passage 205 can be formed with the elongate flexible shaft having a relatively uniform inner diameter, along its length, within a range from about 1 mm to about 8 mm, or within a range of about 1.5 mm to about 3 mm. The shaft 204 can include a piston 214 disposed within the passage 205. The piston 214 can be sized and shaped to occlude the passage at a distal portion 216 of the flexible shaft. The piston 214 can be arranged to travel toward or away from the distal end 212 of the shaft 204 while maintaining a fluid seal, within the passage, between first and second sides of the piston. The piston 214 can move via a pressure change received at the second aperture 208. For example, a positive pressure can force the piston toward the distal end 212 of the shaft 204. In an example, a vacuum or low-pressure environment can move the piston toward the fitting or a first end 218 of the flexible shaft 204.

FIG. 2B depicts an example of an endobronchial check valve placement device paired with a source of external pressure. For example, the source of external pressure can be a pump 220, such as a syringe, sized and shaped to pair with the fitting 202 at or near the first aperture 206, such as at a Leur connector or other fluid-tight connector at a proximal end of the fitting. In an example, the pump 220 can include a first fluid such as a saline solution, sterile water, a medical hydraulic fluid, or another biocompatible fluid. Following coupling of the pump 220 and the endobronchial check valve placement device, the first fluid can be confined within a first liquid hydraulic circuit defined between the pump and the valve 210 of the fitting 202 while the valve is in the closed position. For example, the pump 220 can include a pump piston 222, and the first liquid hydraulic circuit can be defined between the pump piston 222 and the valve 210 of the fitting 202 while the valve 210 is in the closed position. The elongate flexible shaft 204 can contain a second fluid, which can be similar in composition as the first fluid. When the valve 210 is in a closed position, as shown in FIG. 2A, the second fluid can be fluidically sealed, such as in a second liquid hydraulic circuit defined within the passage 205 and between the piston 214 and the second aperture 208 of the fitting 202. When the valve 210 is moved toward an open position, as shown in FIG. 2B, the second fluid and the first fluid (and the first liquid hydraulic circuit and the second liquid hydraulic circuit) can be fluidly connected across the valve 210, such as within a cavity of the fitting 202. Following fluid connection of the first fluid and the second fluid, positive pressure received by the endobronchial check valve placement device 200 can be translated, e.g., hydraulically, to movement of the piston 214 toward the distal end 212 of the shaft 204. In another example, the first and second fluids can each be gaseous in composition and the pump 220 can manipulate the piston 214 generally pneumatically. In another example, the endobronchial check valve placement device 200 can receive only the first fluid from the pump 220, while the valve 210 is in a non-closed position, to move the piston 214 toward the distal end 212 of the shaft 204.

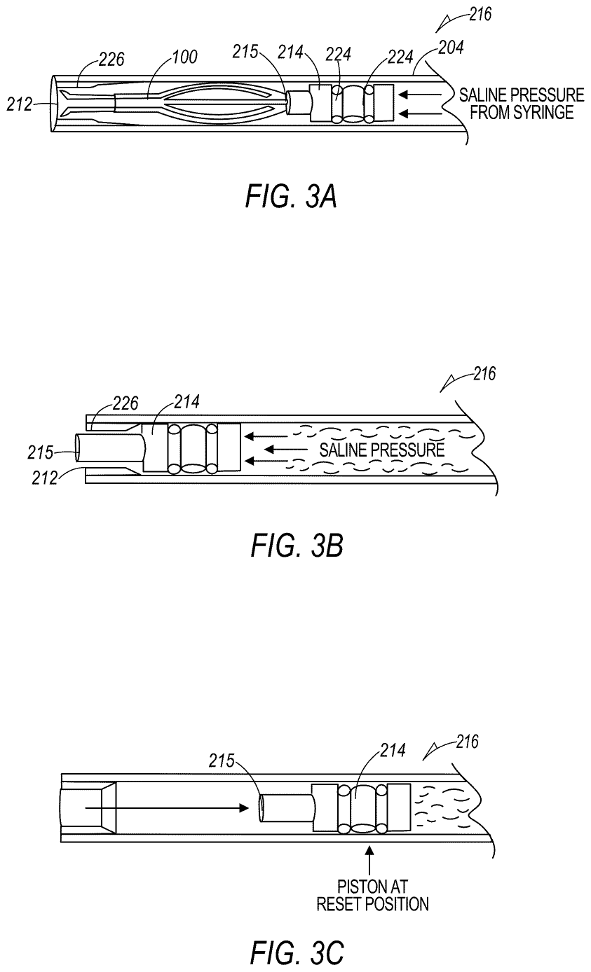

FIG. 3A, FIG. 3B, and FIG. 3C each depict an example of the distal portion 216 of the elongate flexible shaft 204 during motion of the piston 214 within the passage 205 of the shaft 204. As depicted in FIG. 3A, the piston 214 can include one or more O-rings 224 to help maintain a seal between the piston 214 and the passage 205 of the shaft 204. Also, the piston 214 can include a piston feature 215, which can be sized and shaped to prevent slipping, twisting, or nonlinear translation of force from the piston 214 to the endobronchial check valve 100. For example, the piston feature 215 can include a concave or angular shape to maintain contact with the endobronchial check valve 100 during deployment and to help keep the endobronchial check valve 100 generally centered at the piston feature 215 until the valve 100 is released and placed. The elongate flexible shaft 204 can include a piston retainer 226 disposed at or near the distal end 212 of the shaft 204. For example, the piston retainer 226 can be a collar ring, a snap ring, a sleeve, a flange, a clip, or other retaining feature arranged to capture the distal end of the piston 214 within the distal portion 216 of the elongate flexible shaft 204. When the elongate flexible shaft 204 is loaded with an endobronchial check valve 100, the piston retainer 226 can be arranged such as to allow the distal traveling of the piston to the endobronchial check valve 100 to be ejected from the distal end 212 of the shaft 204 while restricting the piston 214 from traversing the distal end 212 of the shaft 204. For example, a reduced inner passage, defined by the piston retainer 226 can allow the endobronchial check valve 100 to be deployed while restricting travel of the piston 214 at the distal end 212 of the shaft 204. Where the piston retainer 226 includes a collar ring, an inner diameter of the collar ring can be smaller than the outer diameter of the piston 214. As depicted in FIG. 3B, positive fluid pressure within the elongate flexible shaft 204 can move the piston toward the distal end 212 of the shaft 204, and thus toward the piston retainer 226.

As depicted in FIG. 3C, after the piston 214 is moved toward the distal end 212 of the shaft 204 to eject or deploy the endobronchial check valve 100, the piston 214 can be replaced, such as moved proximally away from the distal end 212 of the shaft 204 in preparation for the elongate flexible shaft to be reloaded for an additional endobronchial implant deployment. The piston 214 can be replaced via suction, e.g., supplied by the external source of pressure or the pump 220 (as depicted in FIG. 2B). The piston 214 can also be replaced via a pusher tool or pin insertable at the distal end 212 of the shaft 204 or via insertion of an additional, different endobronchial check valve or other endobronchial implant at the distal end 212. For example, an additional endobronchial check valve can be inserted into the elongate flexible shaft 204 via a valve loading device. An example of a valve loading device is described in US Patent Publication Serial No. US 2014/0277574A1, which is incorporated by reference herein in its entirety for teaching of devices, systems, and methods for valve loading into a deployment device.

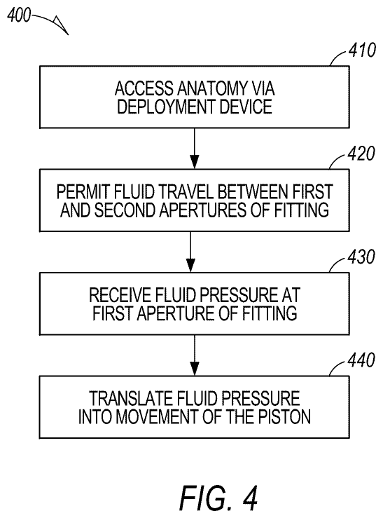

FIG. 4 is a flowchart describing a method of placing an endobronchial check valve via a deployment device.

At 410, anatomy within a lung of a patient can be accessed via a distal end of a deployment device. Here, the deployment device can include a fitting at a proximal end of the deployment device that includes first and second apertures and a valve configured to selectively restrict fluid travel therebetween, an elongate flexible shaft extending toward the distal end of the deployment device and defining a passage between the second aperture and a distal end of the shaft, the shaft including a piston disposed within the passage at a distal portion of the flexible shaft.

At 420, fluid travel can be selectively permitted between the first and second apertures of the fitting. For example, the valve of the fitting can be actuated, manipulated, or operated, such as moved toward an open position, to permit fluid travel between the first and second apertures. In an example, liquid can be confined, e.g., via the valve arranged in a non-open position, to a first liquid hydraulic circuit established between the first aperture of the fitting and the valve. Here, the confining of the liquid can include maintaining a fluid seal between an internal cavity of the fitting and each of the passages and an external ambient environment. As such, the selectively permitting fluid travel between the first and second apertures of the fitting can connect the first liquid hydraulic circuit to a second liquid hydraulic circuit between the valve and the piston.

At 430, fluid pressure can be received at the first aperture of the fitting. For example, a pump or a syringe can be operated or actuated to apply a positive pressure to the first aperture. In an example, a pump piston or plunger can be moved toward a distal end of the pump to force a fluid contained therein toward the first aperture of the fitting.

At 440, the fluid pressure can be translated into movement of the piston within the passage toward the distal end of the shaft. For example, the method can include ejecting, via the piston, an endobronchial check valve from the distal end of the elongate flexible shaft and into the anatomy within the lung of the patient. The method can also include restricting, via a piston retainer, the piston from traversing the distal end of the shaft during the ejecting of the endobronchial check valve.

The following, non-limiting examples, detail certain aspects of the present subject matter to solve the challenges and provide the benefits discussed herein, among others.

Example 1 is a device for bronchoscopy, the device comprising: a fitting including a first aperture, a second aperture, and a valve selectively engageable to restrict fluid travel between the first and second apertures; an elongate flexible shaft sized and shaped to be fluidly coupled, at a proximal end of the shaft, with the second aperture of the fitting, the elongate flexible shaft defining a passage between the second aperture and a distal end of the shaft; and a piston sized and shaped to occlude the passage at a distal portion of the flexible shaft, the piston configured to travel toward the distal end of the shaft while maintaining a fluid seal, within the passage, between first and second sides of the piston; wherein upon the valve being selectively engaged to allow fluid travel between the first and second apertures, fluid pressure received at the first aperture of the fitting, via an external source of fluid pressure, is brought into fluid communication with the piston to drive the piston toward the distal end of the shaft.

In Example 2, the subject matter of Example 1 includes, wherein selective engagement of the valve, included in the fitting to restrict fluid travel between the first and second apertures, confines liquid to a first liquid hydraulic circuit established between the first aperture and the valve.

In Example 3, the subject matter of Examples 1-2 includes, wherein the elongate flexible shaft includes a piston retainer configured to: allow the traveling of the piston to force an endobronchial implant, disposed within the elongate flexible shaft between the piston and the distal end of the shaft, to be ejected from the distal end of the shaft; and restrict the piston from traversing the distal end of the shaft.

In Example 4, the subject matter of Example 3 includes, wherein the piston retainer is configured to allow traveling of the piston to force the endobronchial implant, including an implantable endobronchial check valve including a nitinol frame, to be ejected from the distal end of the shaft.

In Example 5, the subject matter of Examples 3-4 includes, wherein the piston retainer includes a collar ring disposed at the distal end of the shaft, wherein an inner diameter of the collar ring is less than an outer diameter of the piston.

In Example 6, the subject matter of Examples 1-5 includes, wherein the first aperture of the fitting is included in a Leur lock connector at a proximal end of the fitting and the second aperture of the fitting is included in a barbed connector at a distal end of the fitting.

In Example 7, the subject matter of Example 6 includes, wherein the external source of fluid pressure includes a syringe configured to pair with the Leur lock connector at the proximal end of the fitting to supply the fluid pressure at the first aperture.

Example 8 is a bronchoscopy system for accessing anatomy within a lung of a patient, the system comprising: a pump including a chamber and a first piston located inside the first chamber; a check valve placement device including: a fitting including a first fitting aperture fluidly connected to the pump, a second fitting aperture, and a fitting valve selectively engageable to restrict fluid travel between the first and second fitting apertures; an elongate flexible shaft sized and shaped to be fluidly coupled, at a proximal end of the shaft, with the second fitting aperture, the elongate flexible shaft defining a passage between the second fitting aperture and a distal end of the shaft; and a second piston sized and shaped to occlude the passage at a distal portion of the flexible shaft, the piston configured to travel toward the distal end of the shaft while maintaining a fluid seal, within the passage, between first and second sides of the second piston; wherein upon the fitting valve being selectively engaged to allow fluid travel between the first and second fitting apertures, the pump is placed in fluid communication with the second piston such that movement of the first piston drives the second piston toward the distal end of the shaft, to eject an implantable endobronchial check valve from the distal end of the elongate flexible shaft.

In Example 9, the subject matter of Example 8 includes, the implantable endobronchial check valve sized and shaped to be disposed within the distal portion of the flexible shaft, the check valve deployable to at least partially occlude an airway within the lung of the patient and to restrict fluid travel across the check valve in a first direction while allowing fluid travel across the valve in a second direction, opposite the first direction.

In Example 10, the subject matter of Example 9 includes, wherein the implantable endobronchial check valve includes a nitinol frame configured to be compressed to a first cross-sectional diameter during disposition within the distal portion of the flexible shaft and to expand to a second cross-sectional diameter via deployment from the distal end of the elongate flexible shaft to at least partially occlude the airway within the lung of the patient.

In Example 11, the subject matter of Example 10 includes, wherein the first cross-sectional diameter is less than three millimeters (mm), and the second cross-sectional diameter is greater than five mm.

In Example 12, the subject matter of Examples 8-11 includes, a first liquid hydraulic circuit established between the pump and the fitting valve; and a second liquid hydraulic circuit established between the second piston and the fitting valve; wherein engaging the fitting valve to allow fluid travel between the first and second fitting apertures fluidly connects the first and second liquid hydraulic circuits.

In Example 13, the subject matter of Examples 8-12 includes, wherein the elongate flexible shaft includes a piston retainer configured to: allow the traveling of the piston to force the endobronchial check valve, disposed within the elongate flexible shaft between the piston and the distal end of the shaft, to be ejected from the distal end of the shaft; and restrict the piston from traversing the distal end of the shaft.

In Example 14, the subject matter of Example 13 includes, wherein the piston retainer is configured to allow traveling of the piston to force the endobronchial check valve including a nitinol frame, to be ejected from the distal end of the shaft.

In Example 15, the subject matter of Examples 13-14 includes, wherein the piston retainer includes a collar ring disposed at the distal end of the shaft, wherein an inner diameter of the collar ring is less than an outer diameter of the piston.

Example 16 is a method for bronchoscopy comprising: accessing anatomy within a lung of a patient via a distal end of a deployment device, the deployment device including: a fitting at a proximal end of the deployment device and including first and second apertures and a valve configured to selectively restrict fluid travel therebetween; and an elongate flexible shaft extending toward the distal end of the deployment device and defining a passage between the second aperture and a distal end of the shaft, the shaft including a piston disposed within the passage at a distal portion of the flexible shaft; selectively permitting, via the valve, fluid travel between the first and second apertures of the fitting; receiving fluid pressure at the first aperture of the fitting; and translating the fluid pressure into movement of the piston within the passage toward the distal end of the shaft.

In Example 17, the subject matter of Example 16 includes, ejecting, via the piston, an implantable endobronchial check valve from the distal end of the elongate flexible shaft and into the anatomy within the lung of the patient.

In Example 18, the subject matter of Example 17 includes, restricting, via a piston retainer, the piston from traversing the distal end of the shaft during the ejecting of the implantable endobronchial check valve.

In Example 19, the subject matter of Examples 16-18 includes, confining liquid, via the valve arranged in a closed position, to a first liquid hydraulic circuit established between the first aperture of the fitting and the valve, wherein confining the liquid includes maintaining a fluid seal between an internal cavity of the fitting and each of the passage and an external ambient environment.

In Example 20, the subject matter of Example 19 includes, wherein the selectively permitting fluid travel between the first and second apertures of the fitting connects the first liquid hydraulic circuit to a second liquid hydraulic circuit between the valve and the piston.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

1The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.1

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device for bronchoscopy, the device comprising:
a fitting including a first aperture, a second aperture, and a valve selectively engageable to restrict fluid travel between the first and second apertures;
an elongate flexible shaft sized and shaped to be fluidly coupled, at a proximal end of the shaft, with the second aperture of the fitting, the elongate flexible shaft defining a passage between the second aperture and a distal end of the shaft; and
a piston sized and shaped to occlude the passage at a distal portion of the flexible shaft, the piston configured to travel toward the distal end of the shaft while maintaining a fluid seal, within the passage, between first and second sides of the piston;
wherein upon the valve being selectively engaged to allow fluid travel between the first and second apertures, fluid pressure received at the first aperture of the fitting, via an external source of fluid pressure, is brought into fluid communication with the piston to drive the piston toward the distal end of the shaft.

2. The device of claim 1, wherein selective engagement of the valve, included in the fitting to restrict fluid travel between the first and second apertures, confines liquid to a first liquid hydraulic circuit established between the first aperture and the valve.

3. The device of claim 1, wherein the elongate flexible shaft includes a piston retainer configured to:
allow the traveling of the piston to force an endobronchial implant, disposed within the elongate flexible shaft between the piston and the distal end of the shaft, to be ejected from the distal end of the shaft; and
restrict the piston from traversing the distal end of the shaft.

4. The device of claim 3, wherein the piston retainer is configured to allow traveling of the piston to force the endobronchial implant, including an implantable endobronchial check valve including a nitinol frame, to be ejected from the distal end of the shaft.

5. The device of claim 3, wherein the piston retainer includes a collar ring disposed at the distal end of the shaft, wherein an inner diameter of the collar ring is less than an outer diameter of the piston.

6. The device of claim 1, wherein the first aperture of the fitting is included in a Leur lock connector at a proximal end of the fitting and the second aperture of the fitting is included in a barbed connector at a distal end of the fitting.

7. The device of claim 6, wherein the external source of fluid pressure includes a syringe configured to pair with the Leur lock connector at the proximal end of the fitting to supply the fluid pressure at the first aperture.

8. A bronchoscopy system for accessing anatomy within a lung of a patient, the system comprising:
a pump including a chamber and a first piston located inside the first chamber; and
a check valve placement device including:
a fitting including:
a first fitting aperture fluidly connected to the pump; and
a second fitting aperture;
an elongate flexible shaft sized and shaped to be fluidly coupled, at a proximal end of the shaft, with the second fitting aperture, the elongate flexible shaft defining a passage between the second fitting aperture and a distal end of the shaft; and
a second piston sized and shaped to occlude the passage at a distal portion of the flexible shaft, the piston configured to travel toward the distal end of the shaft while maintaining a fluid seal, within the passage, between first and second sides of the second piston;
wherein the fitting valve is configured to allow fluid travel between the first and second fitting apertures, and the pump is thereby placed in fluid communication with the second piston such that movement of the first piston drives the second piston toward the distal end of the shaft, to eject an implantable endobronchial check valve from the distal end of the elongate flexible shaft.

9. The bronchoscopy system of claim 10, wherein:
the fitting includes a fitting valve selectively engageable to restrict fluid travel between the first and second fitting apertures;
during the fitting valve being selectively engaged to allow fluid travel between the first and second fitting apertures, the pump is placed in fluid communication with the second piston such that movement of the first piston drives the second piston toward the distal end of the shaft.

10. The bronchoscopy system of claim 8, comprising the implantable endobronchial check valve sized and shaped to be disposed within the distal portion of the flexible shaft, the check valve deployable to at least partially occlude an airway within the lung of the patient and to restrict fluid travel across the check valve in a first direction while allowing fluid travel across the valve in a second direction, opposite the first direction.

11. The bronchoscopy system of claim 10, wherein the implantable endobronchial check valve includes a nitinol frame configured to be compressed to a first cross-sectional diameter during disposition within the distal portion of the flexible shaft and to expand to a second cross-sectional diameter via deployment from the distal end of the elongate flexible shaft to at least partially occlude the airway within the lung of the patient.

12. The bronchoscopy system of claim 11, wherein the first cross-sectional diameter is less than 3 millimeters (mm), and the second cross-sectional diameter is greater than 5 mm.

13. The bronchoscopy system of claim 8, comprising:
a first liquid hydraulic circuit established between the pump and the fitting valve; and
a second liquid hydraulic circuit established between the second piston and the fitting valve;
wherein engaging the fitting valve to allow fluid travel between the first and second fitting apertures fluidly connects the first and second liquid hydraulic circuits.

14. The bronchoscopy system of claim 8, wherein the elongate flexible shaft includes a piston retainer configured to:
allow the traveling of the piston to force the endobronchial check valve, disposed within the elongate flexible shaft between the piston and the distal end of the shaft, to be ejected from the distal end of the shaft; and
restrict the piston from traversing the distal end of the shaft.

15. The bronchoscopy system of claim 14, wherein the piston retainer is configured to allow traveling of the piston to force the endobronchial check valve including a nitinol frame, to be ejected from the distal end of the shaft.

16. The bronchoscopy system of claim 14, wherein the piston retainer includes a collar ring disposed at the distal end of the shaft, wherein an inner diameter of the collar ring is less than an outer diameter of the piston.

17. A method for bronchoscopy comprising:
accessing anatomy within a lung of a patient via a distal end of a deployment device, the deployment device including:
a fitting at a proximal end of the deployment device and including first and second apertures and a valve configured to selectively restrict fluid travel therebetween; and
an elongate flexible shaft extending toward the distal end of the deployment device and defining a passage between the second aperture and a distal end of the shaft, the shaft including a piston disposed within the passage at a distal portion of the flexible shaft;
selectively permitting, via the valve, fluid travel between the first and second apertures of the fitting;
receiving fluid pressure at the first aperture of the fitting; and
translating the fluid pressure into movement of the piston within the passage toward the distal end of the shaft.

18. The method of claim 17, comprising ejecting, via the piston, an implantable endobronchial check valve from the distal end of the elongate flexible shaft and into the anatomy within the lung of the patient.

19. The method of claim 18, comprising restricting, via a piston retainer, the piston from traversing the distal end of the shaft during the ejecting of the implantable endobronchial check valve.

20. The method of claim 17, comprising confining liquid, via the valve arranged in a closed position, to a first liquid hydraulic circuit established between the first aperture of the fitting and the valve, wherein confining the liquid includes maintaining a fluid seal between an internal cavity of the fitting and each of the passage and an external ambient environment.
